(19) United States
(12) Patent Application Publication
(10) Pub. No.: US 2015/0327816 A1
(43) Pub. Date: Nov. 19, 2015

(54) X-RAY CT APPARATUS AND CONTROL METHOD

(71) Applicants: Kabushiki Kaisha Toshiba, Minato-ku (JP); Toshiba Medical Systems Corporation, Otawara-shi (JP)

(72) Inventors: Tsunenori KAKINUMA, Otawara (JP); Kazuaki MAEZAWA, Otawara (JP); Takeo NABATAME, Otawara (JP); Osamu MIYASHITA, Otawara (JP); Hiroshi TAKANEZAWA, Otawara (JP); Tatsuro Suzuki, Utsunomiya (JP)

(73) Assignees: Kabushiki Kaisha Toshiba, Minato-ku (JP); Toshiba Medical Systems Corporation, Otawara-shi (JP)

(21) Appl. No.: 14/807,372

(22) Filed: Jul. 23, 2015

Related U.S. Application Data
(63) Continuation of application No. PCT/JP2014/051776, filed on Jan. 28, 2014.

(30) Foreign Application Priority Data
Jan. 31, 2013 (JP) 2013-017187
Jan. 27, 2014 (JP) 2014-012715

(51) Int. Cl. A61B 6/02; A61B 6/03; A61B 6/04

(52) U.S. Cl. CPC A61B 6/027; A61B 6/0457; A61B 6/0407; A61B 6/032

(57) ABSTRACT

According to an embodiment, an X-ray CT apparatus comprises a bed reciprocating member, setting circuitry, determination circuitry, and control circuitry. The bed reciprocating member reciprocally moves the top along the body axis direction of the object. The setting circuitry sets an imaging range in the above helical scanning. The determination circuitry determines whether the position of the top coincides with a planned scan end position when the helical scanning reaches one end of the set imaging range in the body axis direction. If the determination result obtained by the determination circuitry indicates incoincidence, the control circuitry controls the bed reciprocating member to move the position of the top to the planned scan end position.

### CROSS-REFERENCE TO RELATED APPLICATIONS

This application is a Continuation Application of PCT application No. PCT/JP2014/051776, filed on Jan. 28, 2014, and is based upon and claims the benefit of priority from Japanese Patent Applications No. 2013-017187, filed on Jan. 31, 2013; and No. 2014-012715, filed on Jan. 27, 2014, the entire contents of all of which are incorporated herein by reference.

### FIELD

Embodiments described herein relate generally to an X-ray CT apparatus and a control method.

### BACKGROUND

Recently, there is available helical scanning as one of scanning schemes using an X-ray CT (Computed Tomography) apparatus. Helical scanning is an imaging technique of continuously executing a unit scan by causing an X-ray tube to make one rotation on a circular orbit centered on an object while continuously reciprocating a top.

In a conventional X-ray CT apparatus capable of executing the helical scanning described above, however, the position of the top at the end time of a scan sometimes shifts from a planned position because of operational irregularity and the like at the time of the reciprocal movement of the top.

For this reason, the start position of the reciprocal movement of the top at time of the execution of the next scan shifts. This leads to a failure to acquire projection data suitable for diagnosis.

It is an object to provide an X-ray CT apparatus and a control method which can correct the positional shift of the top at the end time of a scan from a planned position, which is caused by operational irregularity and the like at the time of the reciprocal movement of the top.

### BRIEF DESCRIPTION OF THE DRAWINGS

FIG. 1 is a schematic view showing an example of the arrangement of an X-ray CT apparatus according to an embodiment.

FIG. 2 is a schematic view for explaining helical scanning by the X-ray CT apparatus according to the same embodiment.

FIG. 3 is a schematic view for explaining a positional shift correction function of the X-ray CT apparatus according to the same embodiment.

FIG. 4 is a flowchart showing an example of the operation of the X-ray CT apparatus according to the same embodiment.

FIG. 5 is a flowchart showing an example of the operation of the X-ray CT apparatus according to a modification of the same embodiment.

FIG. 6 is a schematic view for explaining the positional shift correction function of the X-ray CT apparatus according to the modification.

FIG. 7 is a schematic view for explaining the rotational angle shift correction function of the X-ray CT apparatus according to the modification.

### DETAILED DESCRIPTION

In general, according to an embodiment, an X-ray CT apparatus continuously executes scanning of causing an X-ray tube to make one rotation on a circular orbit centered on an object placed on the top, and acquires projection data of the object by performing helical scanning of continuously reciprocally moving the top.

The above X-ray CT apparatus includes a bed reciprocating member, setting circuitry, determination circuitry, and control circuitry.

The bed reciprocating member reciprocally moves the top along the body axis direction of the object.

The setting circuitry sets an imaging range in the above helical scanning.

The determination circuitry determines whether the position of the top coincides with a planned scan end position when the helical scanning reaches one end of the set imaging range in the body axis direction.

If the determination result obtained by the determination circuitry indicates incoincidence, the control circuitry controls the bed reciprocating member to move the position of the top to the planned scan end position.

An X-ray CT apparatus and its program according to each embodiment will be described below with reference to the accompanying drawings. The following X-ray CT apparatus can be implemented by either a hardware arrangement or a composite arrangement of hardware resources and software. As software as a composite arrangement, there is used a program which is installed in a computer in advance via a network or storage medium and causes the computer to implement each function of the X-ray CT apparatus.

Note that the X-ray CT apparatus includes various types of apparatuses, e.g., a rotate/rotate-type apparatus in which the X-ray tube and the X-ray detector rotate together around an object, and a stationary/rotate-type apparatus in which many X-ray detection elements arrayed in the form of a ring are fixed, and only the X-ray tube rotates around an object. Either type can be applied to each embodiment. Recently, with advances toward the commercialization of a so-called multi-tube type X-ray CT apparatus having a plurality of pairs of X-ray tubes and X-ray detectors mounted on a rotating frame, related techniques have been developed. The present invention can be applied to both a conventional single-tube type X-ray CT apparatus and a multi-tube type X-ray CT apparatus in each embodiment described below. The single-tube, rotate/rotate-type X-ray CT apparatus will be exemplified here.

A case in which helical scanning is used as an imaging technique will be mainly described below. Helical scanning is an imaging technique of continuously reciprocating the top while continuously executing a scanning operation of causing the X-ray tube to make one rotation on a circular orbit centered on the object placed on the top. Note that this helical scanning is also called helical shuttle scanning. In addition, in this helical scanning operation, it is necessary to perform a scan (to be written as a stationary scan hereinafter) while the top is stationary at an end portion of the reciprocal movement range of the top in addition to helical scanning performed during the movement of the top. This stationary scan is performed to acquire projection data for reconstructing a tomographic image at a position outside the reciprocal movement range of the top.

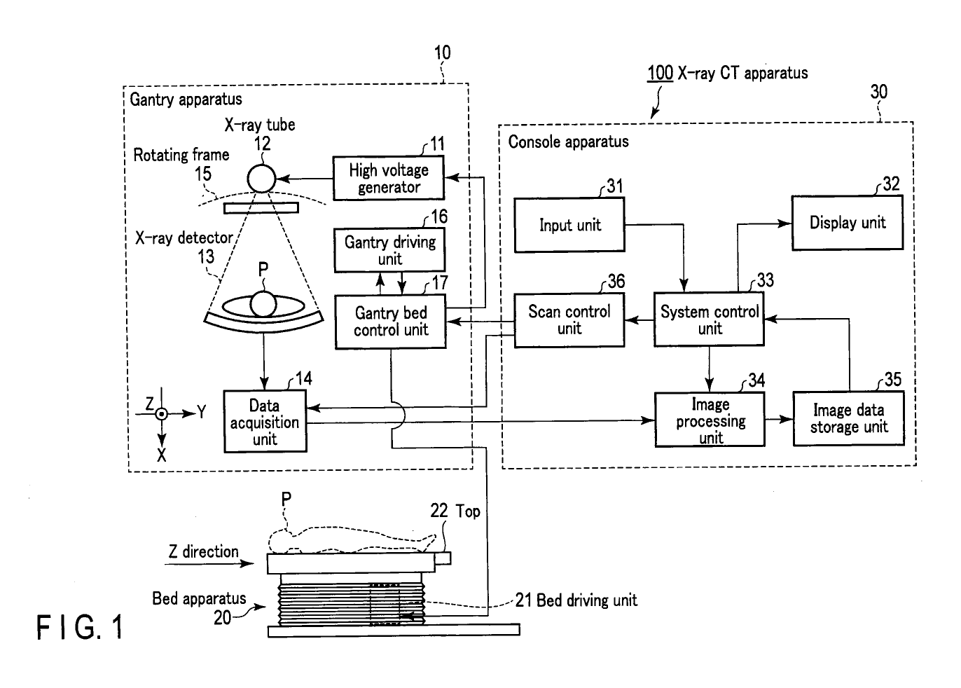

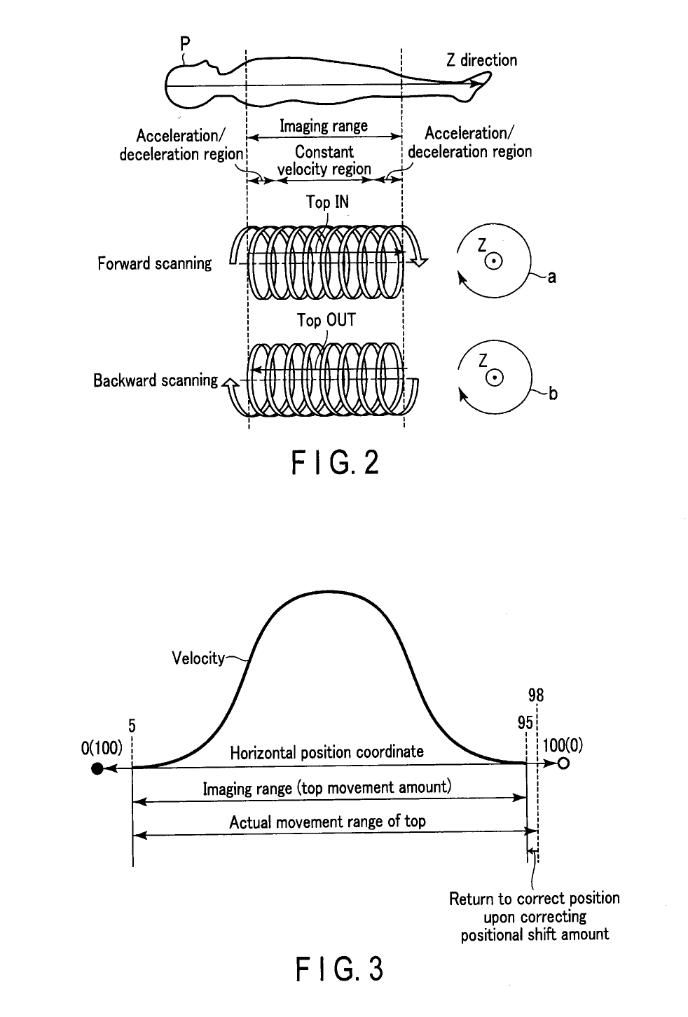

FIG. 1 is a schematic view showing an example of the arrangement of the X-ray CT apparatus according to an embodiment. FIG. 2 is a schematic view for explaining helical scanning performed by the X-ray CT apparatus according to the same embodiment. An X-ray CT apparatus 100 shown in FIG. 1 includes a gantry apparatus 10, a bed apparatus 20, and a console apparatus 30. The functions of the apparatuses 10, 20, and 30 constituting the X-ray CT apparatus 100 will be described in detail below.

As shown in FIG. 1, the gantry apparatus 10 is equipped with an annular or disk-like rotating frame 15. The rotating frame 15 supports an X-ray tube 12 and an X-ray detector 13 so as to allow them to rotate about a rotation axis. The rotating frame 15 supports the X-ray tube 12 and the X-ray detector 13 so as to make them face each other through an object P. The rotating frame 15 is normally connected to a gantry driving unit 16.

The gantry driving unit 16 continuously rotates the rotating frame 15 under the control of a gantry bed control unit 17. At this time, the X-ray tube 12 and the X-ray detector 13 supported on the rotating frame 15 rotate about the rotation axis. That is, the gantry driving unit 16 rotates the X-ray tube 12 and the X-ray detector 13 around the object P. In addition, the gantry driving unit 16 detects the rotational angle of the X-ray tube 12. The detected rotational angle is sent to the gantry bed control unit 17. Note that the detection of a rotational angle may be executed by using, for example, an encoder which converts the rotational angle displacement of the rotation axis into a pulse signal and an arithmetic circuit which computes a rotational angle based on the number of pulse signals.

The X-axis, the Y-axis, and the Z-axis shown in FIG. 1 will be described below. The Z-axis is an axis defined by the rotation axis of the rotating frame 15. The Y-axis is an axis defined by an axis which is perpendicular to the Z-axis and connects the X-ray focal point of the X-ray tube 12 to the center of the detection surface of the X-ray detector 13. The X-axis is an axis defined by an axis perpendicular to the Y-axis and the Z-axis. As described above, the XYZ orthogonal coordinate system forms a rotating coordinate system which rotates with the rotation of the X-ray tube 12.

The X-ray tube 12 generates a cone X-ray beam upon reception of a high voltage supplied from a high voltage generator 11. The high voltage generator 11 applies a high voltage to the X-ray tube 12 under the control of the gantry bed control unit 17.

The X-ray detector 13 detects the X-rays generated from the X-ray tube 12 and transmitted through the object P. The X-ray detector 13 generates a current signal corresponding to the intensity of the detected X-rays. As the X-ray detector 13, a detector of a type called an area detector or multi-row detector is preferably used. The X-ray detector 13 of this type includes a plurality of X-ray detection elements arrayed two-dimensionally. Assume that in the following description, a single X-ray detection element forms a single channel. For example, 100 X-ray detection elements are one-dimensionally arrayed in an arc direction (channel direction) centered on an X-ray focal point, with the distance from the center to the center of the light-receiving unit of each X-ray detection element being a radius. A plurality of X-ray detection elements arrayed along the channel direction will be referred to as X-ray detection element arrays hereinafter. For example, 64 X-ray detection element arrays are arrayed along the slice direction indicated by the Z-axis. A data acquisition unit (DAS: Data Acquisition System) 14 is connected to the X-ray detector 13.

As mechanisms of converting incident X-rays into charges, the following techniques are the mainstream: an indirect conversion type that converts X-rays into light through a phosphor such as a scintillator and converts the light into charges through photoelectric conversion elements such as photodiodes, and a direct conversion type that uses generation of electron-hole pairs in a semiconductor such as selenium by X-rays and migration of the electron-hole pairs to an electrode, i.e., a photoconductive phenomenon. As an X-ray detection element, either of these schemes can be used.

The data acquisition unit 14 reads out an electrical signal for each channel from the X-ray detector 13 under the control of a scan control unit 36. The data acquisition unit 14 then amplifies the readout electrical signal. The data acquisition unit 14 generates projection data by converting the amplified electrical signal into a digital signal. Note that the data acquisition unit 14 can also generate projection data by reading out an electrical signal from the X-ray detector 13 during a period in which no X-ray irradiation is performed. The generated projection data is supplied to the console apparatus 30 via a noncontact data transmission unit (not shown).

The gantry bed control unit 17 controls the gantry driving unit 16 and a bed driving unit 21 under the control of the scan control unit 36. The gantry bed control unit 17 also controls the bed driving unit 21 to move the position of a top 22 in response to the rotational angle detected by the gantry driving unit 16 as a trigger. In addition, the gantry bed control unit 17 records the rotational angle detected by the gantry driving unit 16 and the position of the top 22 which is moved by the bed driving unit 21 in association with each other.

The bed apparatus 20 is installed near the gantry apparatus 10. The bed apparatus 20 includes the top 22 and the bed driving unit 21. The object P is placed on the top 22. The bed driving unit 21 drives the top 22 under the control of the gantry bed control unit 17 in the gantry apparatus 10. More specifically, as shown in FIG. 2, the bed driving unit 21 moves the top 22 at a constant velocity in a constant velocity region set in an imaging range. In addition, as shown in FIG. 2, the bed driving unit 21 accelerates or stops the movement of the top 22 in an acceleration/deceleration region in the imaging range. That is, as shown in FIG. 2, the bed driving unit 21 decelerates and stops the top 22 in the deceleration region. After the top 22 stops, the bed driving unit 21 reverses the moving direction of the top 22. The bed driving unit 21 then accelerates the movement of the top 22 in the acceleration region. This series of operations is repeatedly and continuously executed. With these operations, the bed driving unit 21 reciprocally moves the top 22 along the body axis direction of the object P.

Helical scanning by the X-ray CT apparatus 100 according to this embodiment will be additionally described with reference to FIG. 2. According to helical scanning, the focal point of the X-ray tube 12 (or the X-ray detector 13) draws a helical orbit with respect to the object P. In addition, as shown in FIG. 2, the body axis direction of the object P which extends from the head of the object P to the feet and is indicated by the arrow is defined as a Z direction. A scan in one direction when the top 22 is moved in the same direction as the Z direction is called a forward scan. In addition, a scan in one direction when the top 22 is moved in a direction opposite to the Z direction is called a backward scan. The arrow corresponding to “top IN” shown in FIG. 2 indicates the direction in which the top 22 is moved in a forward scan. The arrow corresponding to “top OUT” shown in FIG. 2 indicates the direction in which the top 22 is moved in a backward scan. Note that arrows indicated by symbols a and b shown in FIG. 2 each indicate the rotating direction of the X-ray tube 12.

As shown in FIG. 1, the console apparatus 30 includes an input unit 31, a display unit 32, a system control unit 33, an image processing unit 34, an image data storage unit 35, and a scan control unit 36.

The input unit 31 is an input interface including a mouse, a keyboard, and a touch panel, and inputs various types of commands and information and the like from the operator to the X-ray CT apparatus 100. For example, the input unit 31 sets or inputs various types of scan conditions in helical scanning in accordance with the operation of the operator. Note that the system control unit 33 stores the respective scan conditions input by the input unit 31 in a memory (not shown) or the like, as needed.

In this case, the scan conditions include, for example, the imaging range of helical scanning in the body axis direction of the object P, the position information of the imaging range, the velocity of the top 22 in helical scanning, a helical pitch, the rotational velocity of the rotating frame 15, and the distance of a constant velocity zone of the top 22. Note that the input unit 31 may further input a range, of the imaging range, in which the top 22 is to be moved at a constant velocity, in accordance with the operation of the operator. In addition, the input unit 31 may further input an angular velocity at which the rotating frame 15 is to be continuously rotated about the rotation axis in accordance with the operation of the operator. Note that the scan control unit 36 may set an angular velocity based on scan conditions.

The display unit 32 is a display such as an LCD (Liquid Crystal Display). The display unit 32 displays medical images stored in the image data storage unit 35, a GUI (Graphical User Interface) for accepting various instructions from the operator, and the like.

The system control unit 33 includes integrated circuits such as an ASIC (Application Specific Integrated Circuit) and an FPGA (Field Programmable Gate Array) and electronic circuits such as a CPU (Central Processing Unit) and an MPU (Micro Processing Unit). More specifically, the system control unit 33 executes overall control on the X-ray CT apparatus 100 by controlling the respective units in the gantry apparatus 10, the bed apparatus 20, and the console apparatus 30. For example, the system control unit 33 controls the image processing unit 34 to reconstruct a medical image based on projection data. In addition, the system control unit 33 outputs various types of scan conditions input via the input unit 31 to the scan control unit 36.

The image processing unit 34 executes various types of processing for the projection data generated by the data acquisition unit 14. More specifically, the image processing unit 34 executes preprocessing such as sensitivity correction for the projection data. The image processing unit 34 reconstructs a medical image based on the reconstruction conditions instructed by the system control unit 33. The image processing unit 34 stores the reconstructed medical image in the image data storage unit 35.

The image data storage unit 35 includes semiconductor memory elements such as a RAM (Random Access Memory), a ROM (Read Only Memory), and a flash memory, a hard disk, and an optical disk. The image data storage unit 35 stores the medical image reconstructed by the image processing unit 34.

The scan control unit 36 includes integrated circuits such as an ASIC and an FPGA and electronic circuits such as a CPU and an MPU. The scan control unit 36 controls the data acquisition unit 14 and the gantry bed control unit 17 based on various types of scan conditions instructed from the system control unit 33. For example, the scan control unit 36 outputs an instruction to rotate the rotating frame 15 to the gantry bed control unit 17 based on scan conditions.

The scan control unit 36 controls the gantry bed control unit 17 to control the high voltage generator 11 so as to reduce exposure of the object P. The gantry bed control unit 17 controls the high voltage generator 11 so as to change X-ray intensity in directions along the Z-axis, the X-axis, and the Y-axis based on a scanogram obtained in advance under the control of the scan control unit 36.

The scan control unit 36 controls the data acquisition unit 14 to acquire projection data. More specifically, the scan control unit 36 controls the data acquisition unit 14 such that the number of views required to reconstruct a tomographic image remains the same at any Z position in forward scanning or backward scanning.

Note that the scan control unit 36 can also calculate a total rotational angle in the constant velocity zone of the top 22 based on the distance of the constant velocity zone as one of various types of scan conditions set or input via the input unit 31 and the rotational velocity of the rotating frame 15 as one of the various types of scan conditions. This allows the scan control unit 36 to calculate the rotational velocity of the X-ray tube 12 at the end position of the constant velocity zone based on the set scan conditions. In addition, the scan control unit 36 can set the rotational angle of the X-ray tube 12 at the scan start position to a predetermined position in forward scanning in the constant velocity zone of the top 22. The scan control unit 36 can calculate the rotational angle of the X-ray tube 12 at the end position of the constant velocity zone in backward scanning in the constant velocity zone of the top 22 in the same manner. With these operations, the scan control unit 36 can decide the relationship (to be referred to as a helical orbit hereinafter) between the rotational angle of the X-ray tube 12 and the position of the top 22 in helical scanning.

In addition, the scan control unit 36 can decide the velocities of the top 22 in forward scanning and backward scanning based on the information of the imaging range of the object P which is input by the input unit 31. Note that the scan control unit 36 can also decide the accelerations of the top 22 in a turn-around portion (to be referred to as the first turn-around portion hereinafter) from forward scanning to backward scanning and in a turn-around portion (to be referred to as the second turn-around portion hereinafter) from backward scanning to forward scanning based on the information of the imaging range. In addition, the scan control unit 36 can decide the velocities of the top 22 in the constant velocity zone in forward scanning and backward scanning based on the information of the imaging range. Furthermore, the scan control unit 36 can control the X-ray tube 12 and the X-ray detector 13 to acquire projection data by irradiating the object P with X-rays in the acceleration and deceleration zones of the top 22 in the first and second turn-around portions.

Note that the scan control unit 36 can also control the gantry bed control unit 17 such that a rotation end angle on a helical orbit at the end position of the forward constant velocity zone coincides with a rotational angle on a helical orbit at the start position of the backward constant velocity zone. In addition, the scan control unit 36 can control the gantry bed control unit 17 such that a rotation end angle on a helical orbit at the end position of the backward constant velocity zone matches a rotation start angle on a helical orbit at the start position of the forward constant velocity zone.

Along with this operation, the scan control unit 36 can match helical orbits in constant velocity zones in a plurality of forward scans. Likewise, the scan control unit 36 can match helical orbits in constant velocity zones in a plurality of backward scans. That is, the scan control unit 36 can implement orbit synchronization scans in constant velocity zones in forward scanning and backward scanning.

The positional shift correction function of the X-ray CT apparatus 100 according to this embodiment will be described below with reference to the schematic view of FIG. 3. The positional shift correction function of the X-ray CT apparatus 100 is mainly implemented by the gantry bed control unit 17 in the gantry apparatus 10. The following will describe various types of processing executed by the gantry bed control unit 17 to implement the positional shift correction function.

When helical scanning reaches one end of the set imaging range, the gantry bed control unit 17 determines whether the position of the top 22 at the end of the scan (to be referred to as the scan end position hereinafter) coincides with the planned position of the top 22 at the end of the scan (to be referred to as the planned scan end position hereinafter). Note that the “scan end time” in this case means the “time when the helical scanning has reached one end”.

More specifically, first of all, when helical scanning reaches one end of a desired imaging range in the forward or backward direction, the gantry bed control unit 17 acquires the horizontal position coordinate of a scan start position and the horizontal position coordinate of a scan end position from the bed driving unit 21. The scan start position indicates the position of the top 22 at the scan start time. The horizontal position coordinate in this case indicates the position of the end portion of the head side of the object P placed on the top 22 assuming that the black circle shown in FIG. 3 represents 0 (start point) and the white circle represents 100 (end point), when, for example, the top 22 moves in the forward direction. In addition, when the top 22 moves in the backward direction, the horizontal position coordinate indicates the position of the end portion of the head side of the object P placed on the top 22 assuming that the white circle shown in FIG. 3 represents 0 (start point) and the black circle represents 100 (end point). Note that the range indicated by the start point and the end point coincides with the range in which the top 22 can reciprocally move. In addition, although the horizontal position coordinate indicates the position of the end portion of the head side of the object P placed on the top 22 in this embodiment, a reference point for grasping the movement of the top 22 is not limited to this.

Subsequently, the gantry bed control unit 17 calculates the horizontal position coordinate of a planned scan position in the scan by adding the top movement amount indicated by an imaging range as one of various types of scan conditions set in advance to the horizontal position coordinate of the scan start position. Thereafter, the gantry bed control unit 17 compares the horizontal position coordinate of the scan end position with the horizontal position coordinate of the planned scan end position to determine whether the scan end position coincides with the planned scan end position. Note that the gantry bed control unit 17 may determine the coincidence/incoincidence of the two coordinates by determining whether the difference between them is 0, instead of comparing them.

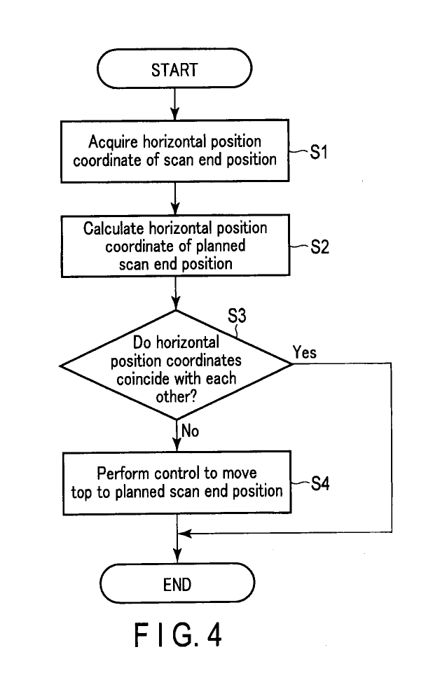

If this determination result indicates coincidence, the gantry bed control unit 17 starts the next helical scanning based on various types of scan conditions set in advance. In addition, if the determination result indicates incoincidence, the gantry bed control unit 17 controls the bed driving unit 21 so as to correct the position of the top 22 based on the planned scan end position. The bed driving unit 21 then moves the top 22 to the planned scan end position (i.e., the start position of the next helical scanning) under the control of the gantry bed control unit 17, as shown in FIGS. 3 and 4.

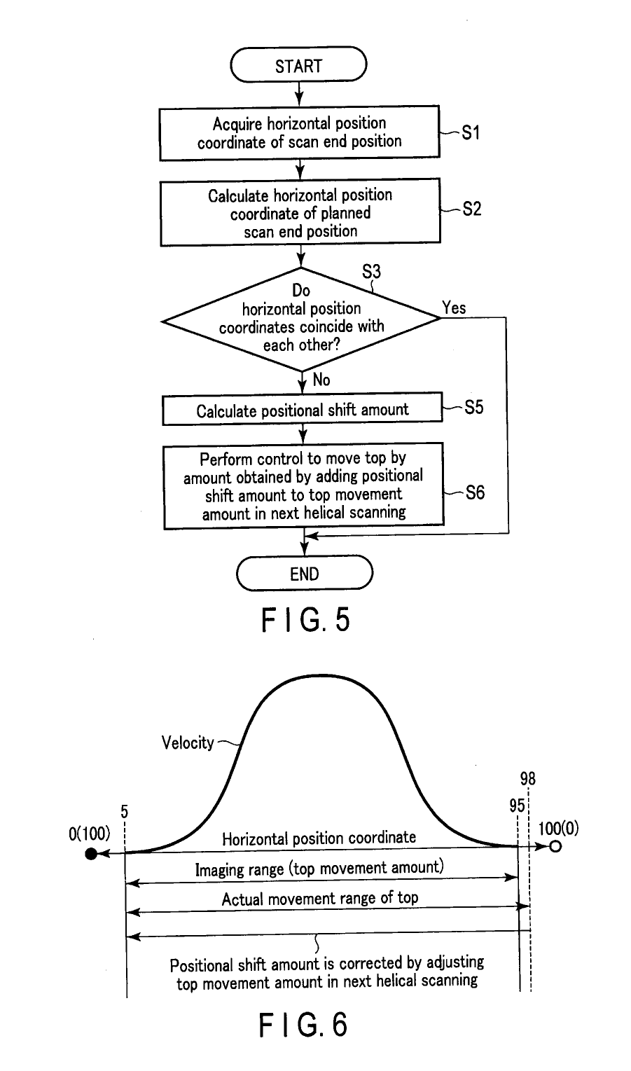

Note that this is not exhaustive, and the bed driving unit 21 may move the top 22 so as to correct a positional shift amount by adjusting a top movement amount in the next helical scanning, as shown in FIGS. 5 and 6, under the control of the gantry bed control unit 17.

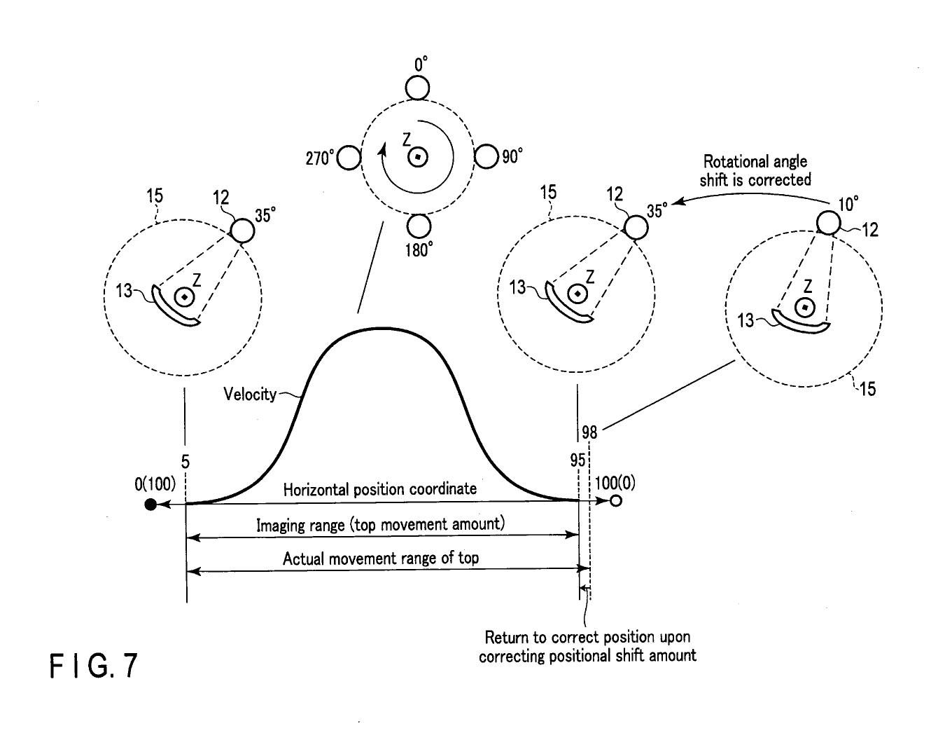

In addition, the bed driving unit 21 may move the top 22 so as to correct a rotational angle shift, as shown in, for example, FIG. 7, under the control of the gantry bed control unit 17.

An example of the operation of the X-ray CT apparatus 100 having the above arrangement will be described next with reference to the schematic view of FIG. 3 and the flowchart of FIG. 4.

First of all, when helical scanning reaches one end of a desired imaging range, the gantry bed control unit 17 acquires the horizontal position coordinate of a scan start position and the horizontal position coordinate of a scan end position from the bed driving unit 21 (step S1). Assume that in this case, the horizontal position coordinate of the scan end position indicates “98”, as shown in FIG. 3.

Subsequently, the gantry bed control unit 17 calculates the horizontal position coordinate of a planned scan end position in this scan by adding the top movement amount indicated by an imaging range as one of various types of scan conditions set in advance to the horizontal position coordinate of the scan start position (step S2). Assume that in this case, the horizontal position coordinate of the scan start position indicates “5”, and the imaging range indicates “5 to 95”, that is, the top movement amount indicates “90 (=95−5)”, as shown in FIG. 3. In this case, the horizontal position coordinate of the planned scan end position is “95 (=5 +90)”. Assume that the horizontal position coordinate of the scan start position is acquired from the bed driving unit 21 in the processing in step S1 described above.

The gantry bed control unit 17 then compares the horizontal position coordinate of the scan end position with the horizontal position coordinate of the planned scan end position to determine whether the scan end position coincides with the planned scan end position (step S3). In this operation, since the horizontal position coordinate of the scan end position indicates “98” and the horizontal position coordinate of the planned scan end position indicates “95”, the process advances to step S4 described later.

Note that if the determination result in step S3 indicates coincidence (YES in step S3), the gantry bed control unit 17 controls the bed driving unit 21 to start the next helical scanning based on various types of scan conditions set in advance.

In addition, if the determination result obtained by the processing in step S3 indicates incoincidence (NO in step S3), the gantry bed control unit 17 controls the bed driving unit 21 so as to move the top 22 to the position coinciding with the horizontal position coordinate of the planned scan end position calculated in step S2. Thereafter, the bed driving unit 21 moves the top 22 to the planned scan end position under the control of the gantry bed control unit 17 (step S4).

After step S4, the gantry bed control unit 17 may control the bed driving unit 21 so as to start moving the top 22 at the next scan start time at the timing when the rotational angle “35°” recorded in association with the planned scan end position “95” coincides with the rotational angle “35°” of the X-ray tube 12, as shown in FIG. 7. In this case, as shown in FIG. 7, a rotational angle shift is corrected. In addition, since synchronization can be established between a plurality of backward scans with respect to the position of the top 22 and the rotational angle of the X-ray tube 12, backward orbit synchronization scanning can be implemented.

Likewise, when performing forward scanning, rotational angle shift correction may be executed in steps S1 to S4 and subsequent steps. In this case, likewise, since synchronization can be established between a plurality of forward scans with respect to the position of the top 22 and the rotational angle of the X-ray tube 12, forward orbit synchronization scanning can be implemented.

The embodiment described above includes the gantry bed control unit 17 which determines whether a scan end position coincides with a planned scan end position when helical scanning reaches one end of a set imaging range, and controls the bed driving unit 21 to move the top 22 to the planned scan end position when the determination result indicates incoincidence. With this arrangement, even if the position of the top at the end time of a scan shifts from a planned position because of operational irregularity and the like at the time of reciprocal movement of the top, it is possible to correct this shift.

In addition, when controlling the bed driving unit 21 to start moving the top 22 at the start time of the next scan after step S4 at the timing when a rotational angle recorded in association with a planned scan end position coincides with the rotational angle of the X-ray tube 12, it is possible to correct the rotational angle shift of the X-ray tube 12 in addition to implementing the above effects, thereby implementing orbit synchronization scanning.

A modification of the embodiment described above will be described below.

### [Modification]

This modification will exemplify a positional shift correction function capable of correcting a positional shift caused by operational irregularity and the like at the time of reciprocal movement of the top 22 when performing the next helical scanning, unlike the embodiment described above.

The gantry bed control unit 17 executes the following processing in addition to the above various types of processing.

If the above determination result indicates incoincidence, the gantry bed control unit 17 calculates the absolute value of the difference (to be written as the positional shift amount hereinafter) between the horizontal position coordinate of a scan end position and the horizontal position coordinate of a planned scan end position. In addition, upon calculating the above positional shift amount, the gantry bed control unit 17 controls the bed driving unit 21 so as to move the top 22 by the movement amount obtained by adding the calculated positional shift amount to the top movement amount indicated by an imaging range as one of various types of scan conditions concerning the next helical scanning at the time of the execution of the next helical scanning. The bed driving unit 21 then moves the top 22 by the movement amount obtained by adding the above positional shift amount to the top movement amount indicated by the preset imaging range at the time of the execution of the next helical scanning.

An example of the operation of the X-ray CT apparatus having the above arrangement will be described with reference to the flowchart of FIG. 5 and the schematic view of FIG. 6. Note that the processing in steps S1 to S3 and the processing to be performed when the determination result obtained by the processing in step S3 indicates coincidence are the same as the operation shown in FIG. 4 described above, and hence a detailed description will be omitted. The processing to be performed when the determination result obtained by the processing in step S3 indicates incoincidence will be described first below.

If the determination result obtained by the processing in step S3 indicates incoincidence (NO in step S3), the gantry bed control unit 17 calculates a positional shift amount based on the horizontal position coordinate of a scan end position and the horizontal position coordinate of a planned scan end position (step S5). Assume that in this case, the horizontal position coordinate of the scan end position and the horizontal position coordinate of the planned scan end position indicate the same values as those in the case shown in FIG. 4 described above, that is, the horizontal position coordinate of the scan end position indicates “98” and the horizontal position coordinate of the planned scan end position indicates “95”. In this case, the positional shift amount is “3 (=|98−95|)”.

Subsequently, the gantry bed control unit 17 controls the bed driving unit 21 so as to move the top 22 at the time of the execution of the next helical scanning by the amount obtained by adding the calculated positional shift amount to the top movement amount indicated by an imaging range as one of various types of scan conditions concerning the next helical scanning. The bed driving unit 21 then moves the top 22 by the amount obtained by adding the positional shift amount to the top movement amount (step S6).

Like the embodiment described above, the above modification of the embodiment can correct the shift between the position of the top at the end time of a scan and a planned position because of operational irregularity and the like at the time of reciprocal movement of the top.

Although several embodiments have been described above, they are merely examples and not intended to limit the scope of the present invention. These embodiments can be implemented in other various forms, and various omissions, replacements, and changes can be made without departing from the spirit of the present invention. These embodiments and their modifications are incorporated in the scope and sprit of the present invention, and are also incorporated in the scope of the invention and its equivalents defined in the appended claims.

1. An X-ray CT apparatus which acquires projection data concerning an object placed on a top by helical scanning performed by continuously reciprocally moving the top while continuously executing scanning of causing an X-ray tube to make one rotation on a circular orbit centered on the object, the apparatus comprising:
a bed reciprocating member configured to reciprocally move the top along a body axis direction of the object;
processing circuitry configured to
set an imaging range of the helical scanning in the body axis direction,
determine whether a position of the top coincides with a planned scan end position, when the helical scanning reaches one end of the set imaging range, and
control the bed reciprocating member so as to correct the position of the top based on the planned scan end position when the determination result based on the planned scan end position indicates incoincidence.

2. The X-ray CT apparatus of claim 1, wherein the bed reciprocating member comprises output circuitry configured to output, to the processing circuitry a horizontal position coordinate of a scan start position which is a horizontal position coordinate, of a position of an end portion of the top, which indicates a position of an end portion of a head side of the object and indicates a position of the top at a start time of a scan, and a horizontal position coordinate of a scan end position which indicates a position of the top when the helical scanning reaches one end,
wherein the processing circuitry is configured
to acquire a horizontal position coordinate of the scan start position and a horizontal position coordinate of the scan end position from the output circuitry,
to calculate a horizontal position coordinate of the planned scan end position by adding a movement amount of the top defined by the set imaging range to the acquired horizontal position coordinate of the scan start position,
to determine whether the acquired horizontal position coordinate of the scan end position coincides with the calculated horizontal position coordinate of the planned scan end position, and
to control the bed reciprocating member to move the position of the top to a position coinciding with the calculated horizontal position coordinate of the planned scan end position when the determination result based on the calculated horizontal position coordinate indicates incoincidence.

3. The X-ray CT apparatus of claim 1, wherein the bed reciprocating member comprises output circuitry configured to output, to the processing circuitry, a horizontal position coordinate of a scan start position which is a horizontal position coordinate, of a position of an end portion of the top, which indicates a position of an end portion of a head side of the object and indicates a position of the top at a start time of a scan, and a horizontal position coordinate of a scan end position which indicates a position of the top when the helical scanning reaches one end,
wherein the processing circuitry is configured
to acquire a horizontal position coordinate of the scan start position and a horizontal position coordinate of the scan end position from the output circuitry,
to calculate a horizontal position coordinate of the planned scan end position by adding a movement amount of the top defined by the set imaging range to the acquired horizontal position coordinate of the scan start position,
to determine whether the acquired horizontal position coordinate of the scan end position coincides with the calculated horizontal position coordinate of the planned scan end position,
to calculate, as a positional shift amount, an absolute value of a difference between the acquired horizontal position coordinate of the scan end position and the calculated horizontal position coordinate of the planned scan end position when the determination result based on the calculated horizontal position coordinate indicates incoincidence, and
to control the bed reciprocating member to move the top by a movement amount obtained by adding the calculated positional shift amount to a movement amount of the top defined by the set imaging range in the next helical scanning when executing the next helical scanning.

4. The X-ray CT apparatus of claim 1, further comprising:
a rotating frame configured to rotate the X-ray tube and the X-ray detector around the object;
a rotational angle detector configured to detect a rotational angle of the X-ray tube;
wherein the processing circuitry is configured
to record the detected rotational angle, and
to control the bed reciprocating member to start moving the top at a timing when a rotational angle recorded in association with the planned scan end position coincides with a rotational angle of the X-ray tube when starting a next scan after control based on the planned scan end position.

5. A control method for an X-ray CT apparatus which acquires projection data concerning an object placed on a top by helical scanning performed by continuously reciprocally moving the top while continuously executing scanning of causing an X-ray tube to make one rotation on a circular orbit centered on the object and comprises a memory and a bed reciprocating member configured to reciprocally move the top along a body axis direction of the object, the method comprising:
writing information concerning an imaging range for the helical scanning in the memory;
determining whether a position of the top coincides with a planned scan end position, when the helical scanning reaches one end of the written imaging range; and
controlling the bed reciprocating member so as to correct the position of the top based on the planned scan end position when the determination result obtained in the determining indicates incoincidence.

6. The control method of claim 5, wherein the bed reciprocating member comprises output circuitry configured to output, to the determining, a horizontal position coordinate of a scan start position which is a horizontal position coordinate, of a position of an end portion of the top, which indicates a position of an end portion of a head side of the object and indicates a position of the top at a start time of a scan, and a horizontal position coordinate of a scan end position which indicates a position of the top when the helical scanning reaches one end,
the determining comprises
acquiring a horizontal position coordinate of the scan start position and a horizontal position coordinate of the scan end position from the output circuitry,
calculating a horizontal position coordinate of the planned scan end position by adding a movement amount of the top defined by the written imaging range to the acquired horizontal position coordinate of the scan start position, and
determining whether the acquired horizontal position coordinate of the scan end position coincides with the calculated horizontal position coordinate of the planned scan end position, and
the controlling comprises controlling the bed reciprocating member to move the position of the top to a position coinciding with the calculated horizontal position coordinate of the planned scan end position when the determination result obtained in the determining based on the acquired horizontal position coordinate and the calculated horizontal position coordinate indicates incoincidence.

7. The control method of claim 5, wherein the bed reciprocating member comprises output circuitry configured to output, to the determining, a horizontal position coordinate of a scan start position which is a horizontal position coordinate, of a position of an end portion of the top, which indicates a position of an end portion of a head side of the object and indicates a position of the top at a start time of a scan, and a horizontal position coordinate of a scan end position which indicates a position of the top when the helical scanning reaches one end,
the determining comprises
acquiring a horizontal position coordinate of the scan start position and a horizontal position coordinate of the scan end position from the output circuitry,
calculating a horizontal position coordinate of the planned scan end position by adding a movement amount of the top defined by the written imaging range to the acquired horizontal position coordinate of the scan start position, and
determining whether the acquired horizontal position coordinate of the scan end position coincides with the calculated horizontal position coordinate of the planned scan end position, and
the controlling comprises
calculating, as a positional shift amount, an absolute value of a difference between the acquired horizontal position coordinate of the scan end position and the calculated horizontal position coordinate of the planned scan end position when the determination result obtained in the determining based on the acquired horizontal position coordinate and the calculated horizontal position coordinate indicates incoincidence, and
controlling the bed reciprocating member to move the top by a movement amount obtained by adding the calculated positional shift amount to a movement amount of the top defined by the set imaging range in the next helical scanning when executing the next helical scanning.

8. The control method of claim 5, wherein
the X-ray CT apparatus comprises
a rotating frame configured to rotate the X-ray tube and the X-ray detector around the object; and
a rotational angle detector configured to detect a rotational angle of the X-ray tube, and
the method comprises
recording the detected rotational angle in association with a position of the top before the determining, and
controlling the bed reciprocating member to start moving the top at a timing when a rotational angle recorded in association with the planned scan end position coincides with a rotational angle of the X-ray tube when starting a next scan after the controlling the bed reciprocating member so as to correct the position of the top.
